(19) United States
(12) Patent Application Publication
(10) Pub. No.: US 2025/0228910 A1
(43) Pub. Date: Jul. 17, 2025

(54) EXTRACT OF MELASTOMA DODECANDRUM LOUR. FOR TREATING ULCERATIVE COLITIS, PREPARATION METHOD THEREFOR AND USE THEREOF

(71) Applicant: Zhejiang Chinese Medical University, Hangzhou (CN)

(72) Inventors: Zhaohuan LOU, Hangzhou (CN); Weizhi LAI, Hangzhou (CN); Yingying WANG, Hangzhou (CN); Xunjie ZHENG, Hangzhou (CN); Ke LI, Hangzhou (CN)

(21) Appl. No.: 19/170,025

(22) Filed: Apr. 3, 2025

(30) Foreign Application Priority Data
Apr. 7, 2024 (CN) 202410406849.2

(51) Int. Cl. A61K 36/185; A61P 1/04

(52) U.S. Cl. CPC A61K 36/185; A61P 1/04; A61K 2236/331; A61K 2236/37; A61K 2236/53; A61K 2236/55

(57) ABSTRACT

An extract of Melastoma dodecandrum Lour. for treating ulcerative colitis (UC), a preparation method therefor and use thereof are provided, relating to the technical field of biomedicine. The method for preparing the extract of Melastoma dodecandrum Lour. of the present disclosure includes: soaking Melastoma dodecandrum Lour. in water, followed by heating to boiling, performing a reflux condensation and filtration, and collecting a filtrate, which constitutes the extract of Melastoma dodecandrum Lour.

### CROSS-REFERENCE TO RELATED APPLICATION

This patent application claims the benefit and priority of Chinese Patent Application No. 202410406849.2 filed with the China National Intellectual Property Administration on Apr. 7, 2024, the disclosure of which is incorporated by reference herein in its entirety as part of the present application.

### REFERENCE TO SEQUENCE LISTING

A computer readable XML file entitled “SequenceListing.xml”, which was created on Mar. 4, 2025, with a file size of about 13,739 bytes, contains the sequence listing for this application, has been filed with this application, and is hereby incorporated by reference in its entirety.

### TECHNICAL FIELD

The present disclosure belongs to the technical field of biomedicine, and in particular relates to an extract of Melastoma dodecandrum lour. for treating ulcerative colitis, a preparation method therefor and the use thereof.

### BACKGROUND

Ulcerative colitis (UC) is a chronic inflammatory bowel disease characterized by symptoms such as diarrhea, abdominal pain, and mucopurulent bloody stool. The incidence of UC is on the rise worldwide. The pathogenic factors of UC are complex, and involves genetic predisposition, defects within the epithelial barrier, immune dysregulation, and environmental factors. The treatment of UC is primarily aimed at achieving rapid alleviation of symptoms and healing of the mucosal barrier, while restoring the overall quality of life for patients. For mild to moderate diseases, 5-aminosalicylic acid (5-ASA) remains the primary treatment option. When treatment with 5-ASA is inadequate, treatment with steroids and immunosuppressants should be added to the next step. However, 5-ASA is associated with serious side effects such as impaired renal function, which has become an obstacle to long-term use. Inhibiting inflammation and promoting healing of damaged mucosa remains a challenging issue in the treatment of UC.

Fibrosis is a pathophysiological mechanism of tissue repair after body injury occurs, resulting in the deposition of excess collagen in the extracellular matrix (ECM). Fibrosis, as a common complication of chronic inflammation, can occur in all organs and tissues. Intestinal fibrosis is a process driven by chronic intestinal inflammation, resulting in an increase in myofibroblast and collagen deposition. Collagen, one of the major components of the ECM synthesized during fibrosis, can promotes specific signaling pathways by binding to integrins and discoid domain receptors (DDRs). The transmission of signals within the ECM is mainly mediated by integrins, which are a group of cell surface receptors that anchor cells to the matrix and transmit mechanical and chemical signals, facilitating cell adhesion and migration.

Fibroblast migration is an important factor in fibrosis. One study has shown that TGF-beta 1 enhances this migration, as TGF-beta 1 regulates the migration, proliferation and differentiation of fibroblasts. Integrin-mediated activation of TGF-beta 1 plays an important role in the healing of the intestinal mucosa and in fibrosis. The calcium-dependent cell surface adhesion molecule N-cadherin is strongly inducible by TGF-beta 1 and plays a key role in the interaction between fibroblasts and ECM, which is necessary for fibroblast migration. Severe intestinal fibrosis can lead to intestinal obstruction, which may require surgical intervention. While fibrosis is more common in Crohn's disease, there is evidence that fibrosis also occurs in UC, and may interfere with treatment. End-stage UC may cause severe fibrosis, with a phenomenon of shortening, stiffness and contraction of the colon, known as “lead pipe” colon. Therefore, the study of techniques and drugs that hinder intestinal fibrosis is critical for the current treatment of UC.

Melastoma dodecandrum Lour., also known as Twelve stamen melastoma, is an ethnomedinical plant widely distributed in southern China. Melastoma dodecandrum Lour. has effects of clearing heat and removing toxicity, promoting blood circulation and stopping bleeding, and the extracts of Melastoma dodecandrum Lour. have effects of anti-inflammation, anti-oxidation, hemostasis, and lowering blood glucose. However, research on the use of extracts of Melastoma dodecandrum Lour. for the treatment of UC is limited.

### SUMMARY

In view of this, an objective of the present disclosure is to provide an extract of Melastoma dodecandrum Lour. for treating ulcerative colitis (UC), a preparation method therefor and use thereof, where the extract of Melastoma dodecandrum Lour. has effects of anti-UC, reducing intestinal epithelial cell apoptosis and alleviating colonic fibrosis.

To achieve the above objective of the present disclosure, the present disclosure provides the following technical solutions.

The present disclosure provides a method for preparing an extract of Melastoma dodecandrum Lour., including: soaking Melastoma dodecandrum Lour. in water, followed by heating to boiling, performing a reflux condensation and filtration, and collecting a filtrate, which constitutes the extract of Melastoma dodecandrum Lour.; the reflux condensation is performed for 0.5 to 1.5 h.

In some embodiments, a mass ratio of the Melastoma dodecandrum Lour. to the water is 1:9 to 11, and the soaking is performed for 25 to 35 min.

In some embodiments, the method further includes: mixing the medicine residue resulting from the filtration with water after the filtration, performing extraction for 40 to 50 min at 100° C., performing another filtration, and then combining filtrates; where a mass ratio of the medicine reside to the water is 1:5 to 9.

The present disclosure further provides an extract of Melastoma dodecandrum Lour. obtained by the above preparation method.

The present disclosure further provides use of the above extract of Melastoma dodecandrum Lour. in the preparation of a medicament for treating UC.

The present disclosure further provides use of the above extract of Melastoma dodecandrum Lour. in the preparation of an anti-intestinal fibrosis medicament.

In some embodiments, the extract of Melastoma dodecandrum Lour. reduces colonic epithelial collagen deposition and intestinal epithelial cell apoptosis.

In some embodiments, the extract of Melastoma dodecandrum Lour. has an effect of down-regulating the abnormally high expression of genes and proteins such as N-cadherin, Integrin A1, Vinculin, TGF-beta 2 and Srcin in colonic tissue.

In some embodiments, the extract of Melastoma dodecandrum Lour. has effects of regulating fibroblast migration, reducing colonic fibrosis and inflammatory damage, and protecting goblet cells and colonic epithelial barrier function.

In some embodiments, the medicament includes the extract of Melastoma dodecandrum Lour. and a pharmaceutically acceptable excipient.

Compared with conventional technology, embodiments of the present disclosure have the following beneficial effects.

The present disclosure provides a method for preparing an extract of Melastoma dodecandrum Lour. for treating UC, including soaking Melastoma dodecandrum Lour. in water, followed by heating to boiling, performing a reflux condensation and filtration, and collecting a filtrate, which constitutes the extract of Melastoma dodecandrum Lour. The extract of Melastoma dodecandrum Lour. of the present disclosure may reduce colonic epithelial collagen deposition and intestinal epithelial cell apoptosis of UC, has effects of down-regulating the abnormally high expression of genes and proteins of N-cadherin, Integrin A1, Vinculin, TGF-beta 2 and Srcin in the colonic tissue of UC, reducing colonic fibrosis and inflammatory damage and protecting colonic epithelial barrier function, and also has effects of inhibiting DSS-induced protein overexpression of Wnt5a/b and beta-Catenin and alleviating colonic fibrosis, thereby achieving the effects of treating UC and alleviating colonic fibrosis.

### BRIEF DESCRIPTION OF THE DRAWINGS

FIGS. 1A-1F show that Melastoma dodecandrum Lour. alleviates DSS-induced colitis in mice; (FIG. 1A) effect of Melastoma dodecandrum Lour. on the body weight of mice; (FIG. 1B) DAI in different groups; (FIG. 1C) colon images in different groups (n=3); (FIG. 1D) colon length in different groups; (FIG. 1E) representative HE staining (magnification, 100×) and AB-PAS staining (magnification, 200×) images of colonic tissues in different groups; yellow arrow indicates mucosal damage (FIG. 1F) histological scores of colonic tissues in different groups (n=6); # indicates a comparison of NC with MC, * indicates a comparison of MC with SASP, DRH and DRL; ##, **p<0.01, ###, ***p<0.001, *****, ****p<0.0001;

FIGS. 2A-2H show that Melastoma dodecandrum Lour. reduces intestinal fibrosis, inflammatory factor expression and apoptosis in UC mice; (FIG. 2A) Masson staining of colonic tissues in different groups (magnification, 200×); (FIG. 2B) Sirius red staining of colonic tissues in different groups (magnification, 200×); (FIG. 2C) colonic IL-1 beta immunohistochemical staining (magnification, 200×); (FIG. 2D) TUNEL fluorescence staining of colonic tissues in different groups (magnification, 200×); (FIGS. 2E-2H) quantitative analysis of positive signals in the above staining (n=3); # indicates a comparison of NC with MC, * indicates a comparison of MC with SASP, DRH and DRL; *, *p<0.05, #*, **p<0.01, ###, ***p<0.001, ###*, ****p<0.0001;

FIGS. 3A-3V show transcriptome sequencing analysis of differentially expressed genes (DEGs) and enrichment pathways in UC mice alleviated with Melastoma dodecandrum Lour.; (FIG. 3A) Pearson correlation coefficient; (FIG. 3B) principal component analysis (PCA); (FIG. 3C) volcano plot of DEGs; (FIG. 3D) clustering heatmap of DEGs; (FIG. 3E) GO enrichment barplot; (FIG. 3F) GO enrichment scatterplot; (FIG. 3G) KEGG enrichment histogram; (FIG. 3H) KEGG enrichment scatterplot; (FIG. 3I) GSEA (gene set enrichment analysis) of KEGG focal adhesion pathway; (FIGS. 3J-3P) gene expression level (n=3); (FIGS. 3Q-3V) qPCR quantification of target genes; # indicates a comparison of NC with MC, * indicates a comparison of MC with SASP, DRH and DRL; *, *p<0.05, #*, **p<0.01, ***, ***p<0.001;

FIGS. 4A-4H show that Melastoma dodecandrum Lour. inhibits intestinal fibrosis by reducing the expression of genes and proteins in focal adhesion and Wnt/beta-Catenin pathways; (FIG. 4A-FIG. 4C) immunohistochemistry of colonic N-cadherin, Integrin Al and Vinculin; (FIG. 4D) fluorescence co-localization of N-cadherin and alpha-SMA in colon; (FIG. 4E) immunohistochemical quantitative analysis (n=3); (FIG. 4F) fluorescence intensity of N-cadherin, and PCC of co-localization of N-cadherin and fibroblasts (yellow arrow); (FIG. 4G) immunofluorescence staining of colon for Wnt5a/b and beta-Catenin; (FIG. 4H) quantitative analysis of Wnt5a/b and beta-Catenin fluorescence expression; # indicates a comparison of NC with MC, * indicates a comparison of MC with SASP, DRH and DRL; *, *p<0.05, **, **p<0.01, ***, ***p<0.001.

### DETAILED DESCRIPTION OF THE EMBODIMENTS

The present disclosure provides a method for preparing an extract of Melastoma dodecandrum Lour., including: soaking Melastoma dodecandrum Lour. in water, followed by heating to boiling, performing a reflux condensation and filtration, and collecting a filtrate, which constitutes the extract of Melastoma dodecandrum Lour.; where the reflux condensation is performed for 0.5 to 1.5 h.

In the present disclosure, a mass ratio of the Melastoma dodecandrum Lour. to the water is preferably 1:9 to 11, more preferably 1:10, and the soaking is performed for preferably 25 to 35 min, more preferably 30 min.

In the present disclosure, the method preferably further includes: after the filtering, mixing the medicine residue resulting from the filtration with water, performing extraction for 40 to 50 min at 100° C., performing another filtration, then combining filtrates, performing recovery under reduced pressure at 50 to 55° C. and −0.08 to-0.1 MPa, and performing concentrating to obtain the extract (1 g crude drug/ml); where the mass ratio of the medicine reside to the water is preferably 1:5 to 9, more preferably 1:8.

The present disclosure further provides an extract of Melastoma dodecandrum Lour. obtained by the above preparation method.

The present disclosure further provides use of the above extract of Melastoma dodecandrum Lour. in the preparation of a medicament for treating UC.

The present disclosure further provides the use of the above extract of Melastoma dodecandrum Lour. in the preparation of an anti-intestinal fibrosis medicament.

The extract of Melastoma dodecandrum Lour. of the present disclosure reduces colonic epithelial collagen deposition and intestinal epithelial cell apoptosis.

The extract of Melastoma dodecandrum Lour. of the present disclosure has an effect of down-regulating the abnormally high expression of genes and proteins such as N-cadherin, IntegrinA1, Vinculin, TGF-beta 2 and Srcin in colonic tissue.

The extract of Melastoma dodecandrum Lour. of the present disclosure has effects of regulating fibroblast migration, reducing colonic fibrosis and inflammatory damage, and protecting goblet cells and colonic epithelial barrier function.

The medicament of the present disclosure includes the extract of Melastoma dodecandrum Lour. and a pharmaceutically acceptable excipient. Regarding the active ingredient in the medicament of the present disclosure, the extract of Melastoma dodecandrum Lour. can be used as the sole active ingredient, and can also be used in combination with other active ingredients having an effect of treating UC. The medicament of the present disclosure includes, but is not limited to, a preparation for injection, an emulsion, an ointment, a granule, powder, and an oral liquid. In the present disclosure, there are no particular limitations on the excipient contained in the medicament, so long as common excipients for medicaments in the art.

The technical solutions in the present disclosure will be described clearly and completely below with reference to the examples of the present disclosure. Apparently, the described examples are merely a part of, rather than all of the embodiments of the present disclosure. Based on the examples of the present disclosure, all other embodiments that can be obtained by those of ordinary skill in the art without creative efforts shall fall within the protection scope of the present disclosure.

Methods in the following examples are all conventional methods, unless otherwise specified.

The materials, reagents, etc. used in the following examples are all commercially available, unless otherwise specified.

Experimental result data involved in the examples described below are expressed as mean±standard deviation (SD). Data analysis is performed using GraphPad 8 software. Comparisons between multiple groups are performed using one-way analysis of variance (ANOVA), and comparison between two group is performed using t-test. P<0.05 indicates a statistically significant difference.

### Example 1

Preparation of the Extract of Melastoma dodecandrum Lour.

Firstly, 100 g of dried Melastoma dodecandrum Lour. were soaked in 1 kg of pure water for 30 min, then heated and boiled, subjected to reflux condensation for 1 h, and filtered. Then the filtrate was collected. Next, 800 g of water was added to the residue for extraction at 100° C. for 45 min. The two collected filtrates were combined, recovered under reduced pressure at 50 to 55° C. and −0.08 to −0.1 MPa, and concentrated to obtain the extract of Melastoma dodecandrum Lour. (1 g crude drug/ml).

### Example 2

### Animal Experiments

### 2.1 Experimental Animals

Male C57BL/6 mice (specific pathogen free (SPF), weighing 20-22 g) were purchased from Shanghai Bikai Keyi Biotechnology Co., Ltd., with license number SCXK (Shanghai) 2018-0006. All mice were housed at the Zhejiang Chinese Medical University Laboratory Animal Research Center, and subjected to a 12 h light-dark cycle at controlled temperature (23° C.±1° C.). All experiments were performed in accordance with ethical requirements (Ethical approval number IACUC 202309-18).

### 2.2 Modeling and Grouping

After 1 week of adaptive feeding, thirty mice were randomly divided into a normal control (NC) group, a model control (MC) group, a sulfasalazine (SASP) group, a high dose Melastoma dodecandrum Lour. (DRH) group and a low dose Melastoma dodecandrum Lour. (DRL) group. The NC group, i.e., normal control, was administered distilled water for 8 days. The MC group, i.e., model control, was administered a 2.5% aqueous dextran sulfate sodium (DSS, MP Biomedicals, UK) solution for free drinking for 8 days. Except for the NC group, all other administration groups were administered the same 2.5% DSS as the MC group. The SASP group, i.e., positive control, was administered a sulfasalazine suspension (SASP, H31020557, Sinepharm, China) for 8 days via gavage at 0.5 g/kg, and the DRH group and the DRL group were administered the extract of Melastoma dodecandrum Lour. prepared in Example 1 for 8 days via gavage at 9.2 g crude drug/kg and 2.3 g crude drug/kg, respectively. The mice were observed daily for body weight, stool character and blood in stool. The disease activity index (DAI) was evaluated with reference to accepted criteria. Eight days after molding and administration, the mice were euthanized and the colonic tissues were collected.

### 2.3 Body Weight, DAI, HE Staining and AB-PAS Staining

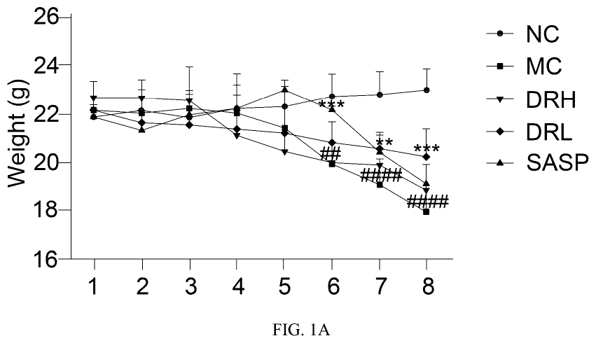

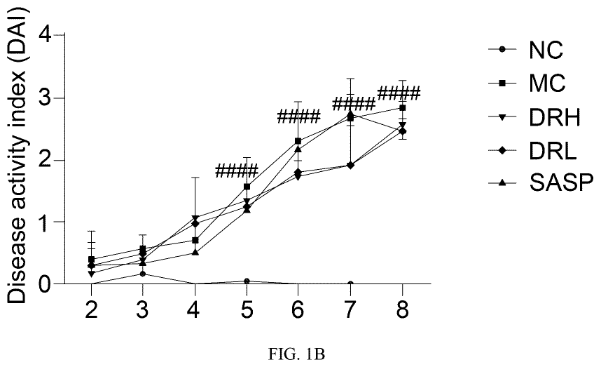

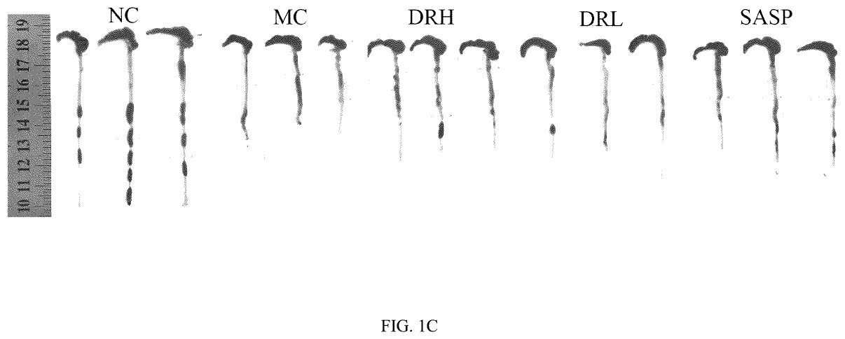

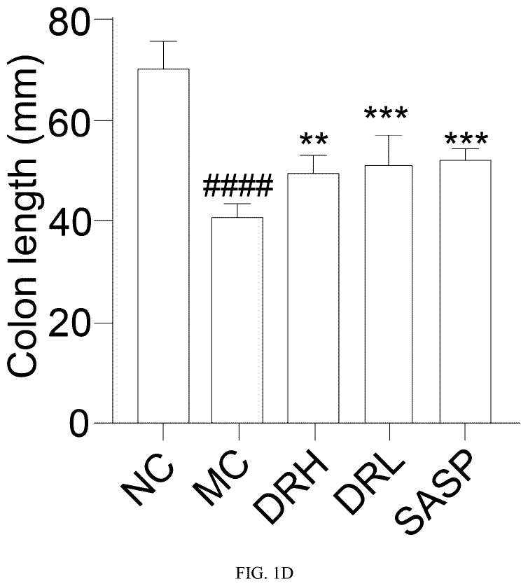

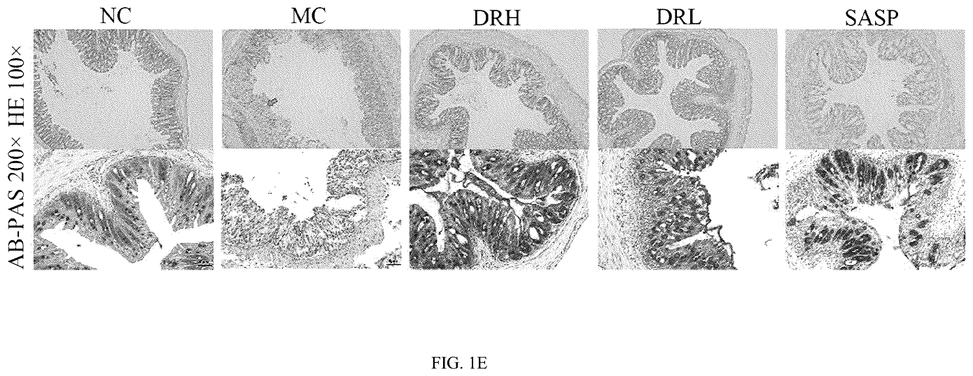

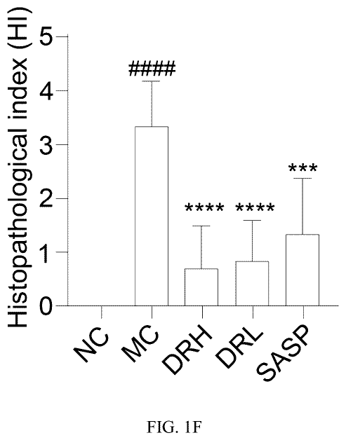

The mice were observed and recorded daily for body weight and DAI score. Hematoxylin and eosin (HE) staining and Alcian Blue-Periodic Acid-Schiff (AB-PAS) staining of the colonic tissue of mice were performed to observe the effect of Melastoma dodecandrum Lour. on DSS-induced colonic tissue damage in UC mice. The specific results are shown in FIG. 1.

The AB-PAS staining method: a portion of colon (about 1 cm in length) was soaked in 4% neutral formaldehyde for 48 h at room temperature for fixation; paraffin sections with a thickness of 4 μm were stained using a hematoxylin-eosin staining kit (HE, D006-1-4, Nanjing Jiancheng Bioengineering Institute, China) and an AB-PAS staining kit (D033-1-1, Nanjing Jiancheng Bioengineering Institute, China); and histopathological examination was performed according to accepted methods, and colonic inflammatory cell infiltration and crypt damage were observed under a light microscope. The cryptal goblet cells appeared blue upon AB-PAS staining.

FIGS. 1A-1F showed that the mice intervened by DSS had a significant weight loss, and had a significant increase in DAI score and a significant decrease in colon length (FIGS. 1A-1D, P<0.0001). HE staining and AB-PAS staining showed that the mice intervened by DSS exhibited typical epithelial defects, inflammatory cell infiltration, crypt damage, and goblet cell depletion (FIG. 1E, as indicated by yellow arrows). The histopathology score in the MC group increased (FIG. 1F). After the mice were intervened by Melastoma dodecandrum Lour., the weight loss was slow, the DAI score decreased (P<0.01), the colon length gradually approached that in the NC group, and the pathological manifestations significantly improved. Compared with treating the mice with DSS, the treatment with Melastoma dodecandrum Lour. significantly reduced the colonic histopathology score (P<0.0001), maintained the integrity of the intestinal epithelium and crypts, and increased the number of goblet cells to maintain mucosal integrity. Overall, these results suggest that Melastoma dodecandrum Lour. can alleviate symptoms and accelerate mucosal healing in UC mice.

### 2.4 Masson Staining, Sirius Red Staining, IL-1 Beta Immunohistochemical Staining, and TUNEL Fluorescent Staining for Apoptosis Detection

The sections of colon were subjected to Masson staining and Sirius red staining to observe colonic collagen deposition and fibrosis. Collagen appeared blue upon Masson staining, and appeared red upon Sirius red staining.

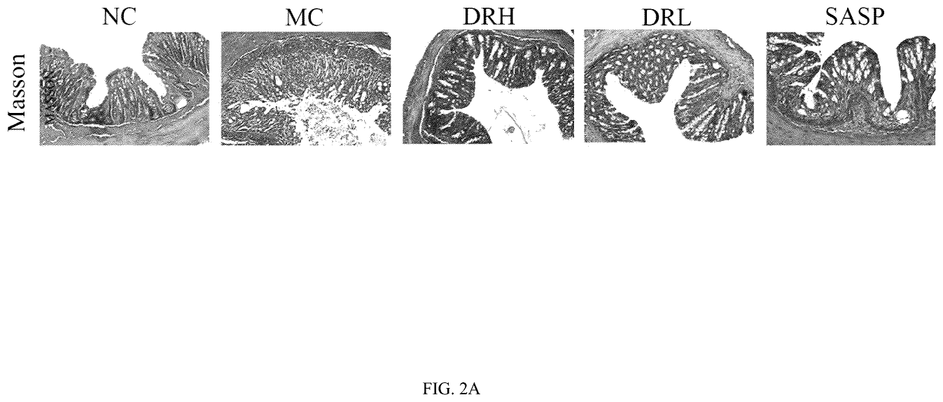

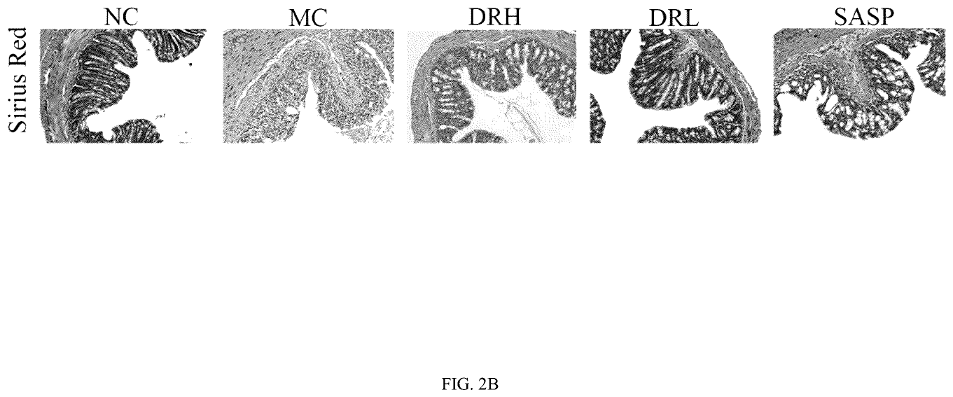

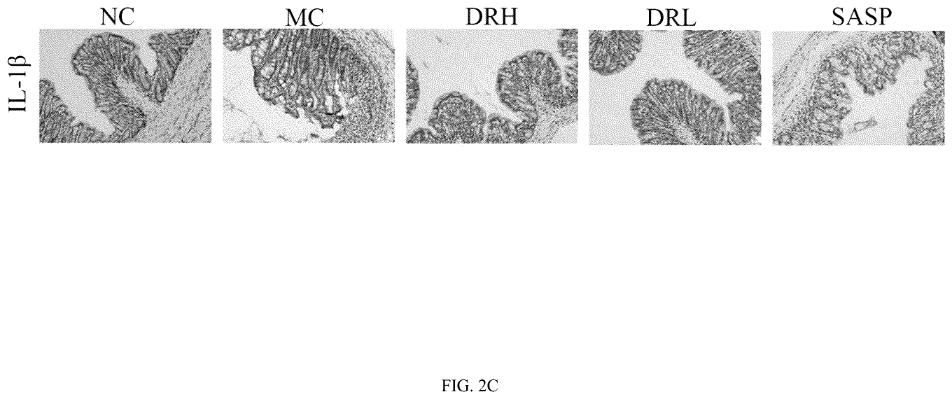

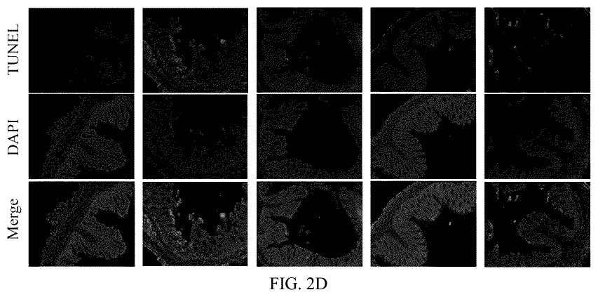

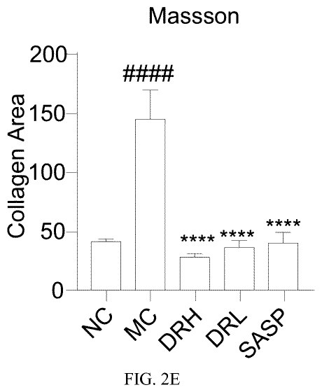

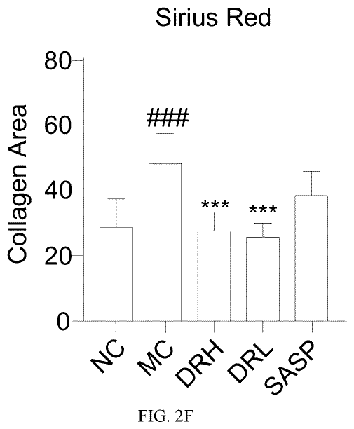

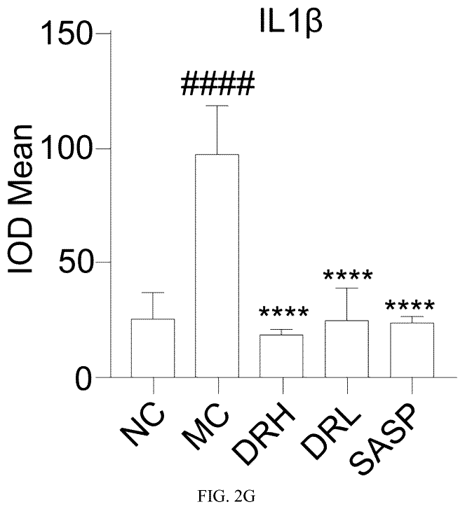

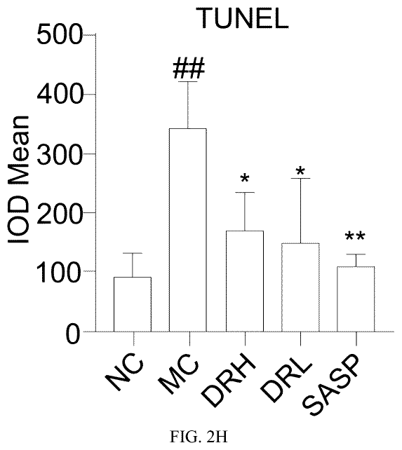

IL-1 beta immunohistochemical staining and terminal deoxynucleotidyl transferase dUTP nick end labeling (TUNEL) fluorescence staining were performed to detect apoptosis. The specific results are as shown in FIGS. 2A-2H.

Masson staining and Sirius red staining: the paraffin sections of colon were deparaffinized and rehydrated; and the distribution of collagen fibers was then observed using a Masson trichrome staining kit (G1340, Solarbio, China) and a Sirius red staining kit (G1472, Solarbio, China). The collagen fibers appeared blue upon Masson staining, and appeared red upon Sirius red staining.

TUNEL fluorescence staining: the paraffin sections of colon were deparaffinized and rehydrated, then repaired in a microwave oven, and washed with phosphated buffered saline (PBS); and the sections were then subjected to fluorescence staining according to the instructions of the TdT-mediated dUTPnick-end labeling (TUNEL) kit (MA0224, meilunbio, China), and mounted with an antifade mounting medium (with 4,6-diamino-2-phenyl indole (DAPI)) (MA0222, meilunbio, China), where the process was protected from light. Under a fluorescence microscope, red fluorescence came from the fragmented DNA of apoptotic cells.

Immunohistochemical staining of IL-1 beta: the paraffin sections of colon were deparaffinized and rehydrated, and then washed with PBS 3 times; then, microwave retrieval was performed using a sodium citrate solution (pH 6.0, P0081, Beyotime, China); an endogenous peroxide was blocked with 3% hydrogen peroxide; blocking was then performed with an immunostaining blocking solution (P0260, Beyotime, China) for 15 min at room temperature; antibody IL1-beta (ET1701-39, Huabio, China; dilution 1:200) was added dropwise, and incubated with the sections overnight at 4° C.; the corresponding HRP secondary antibody (PV-6001, ZSbio, China) was incubated with the sections for 30 min at 37° C., and then washed with PBS; color development was performed with diaminobenzidine (DAB) (ZLI-9019, ZSbio, China), and the sections were subjected to hematoxylin staining, and then rapidly dehydrated in absolute ethanol; and finally, the sections were mounted with neutral resin. Images were taken by an optical microscope, and analyzed using Image-Pro Plus 6.0 software.

It was shown that there was a large amount of collagen deposition in the mucosal and submucosal layers in the MC group (FIG. 2A, 2B 2E, and 2F). The expression of IL-1 beta in the model group increased, and significantly decreased after the treatment with Melastoma dodecandrum Lour., demonstrating that Melastoma dodecandrum Lour. has the effect of improving DSS-induced colonic inflammation in mice (FIGS. 2C and 2G, P<0.0001). At the same time, the TUNEL staining resulted in red fluorescence in the fragmented DNA, and the results showed a large amount of apoptosis in the intestinal epithelium and crypt base in the MC group (FIGS. 2D and 2H). The treatment with Melastoma dodecandrum Lour. significantly reduced collagen deposition, intestinal inflammatory cytokine IL-1 beta expression and apoptosis.

### 2.5 Analysis of DEGs and Enriched Pathways Using Transcriptome Sequencing

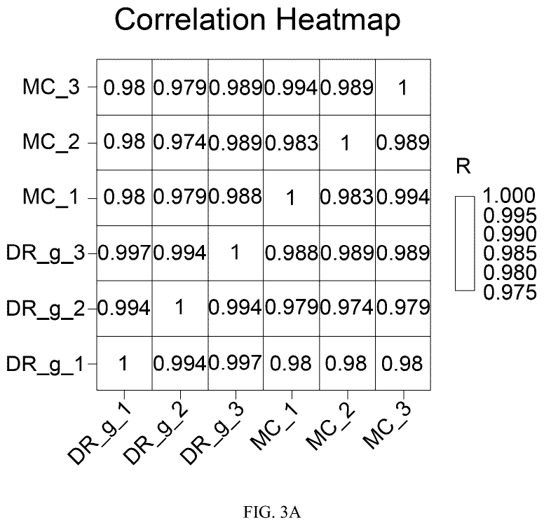

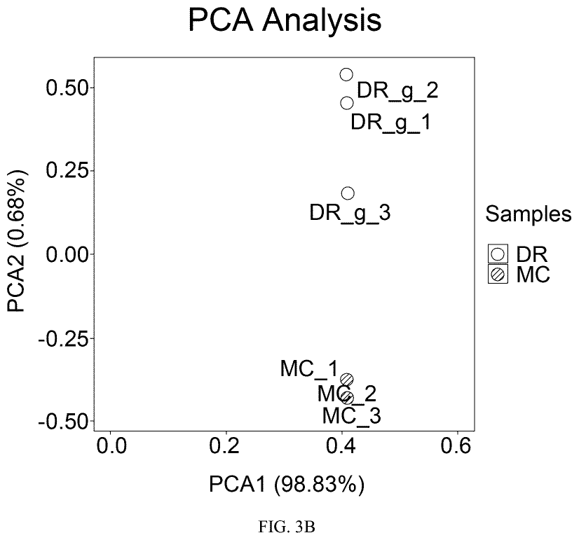

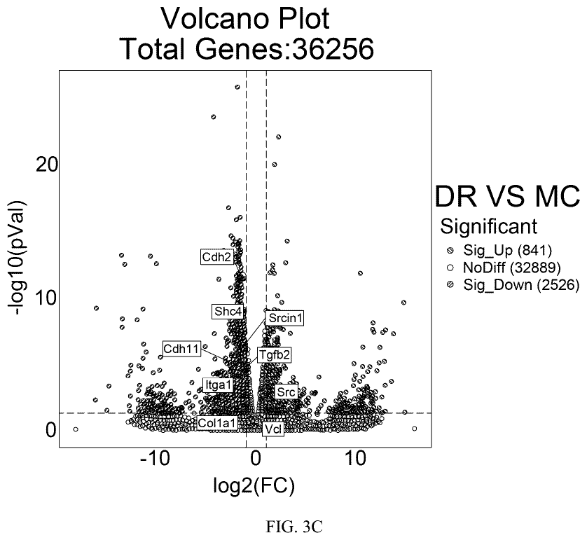

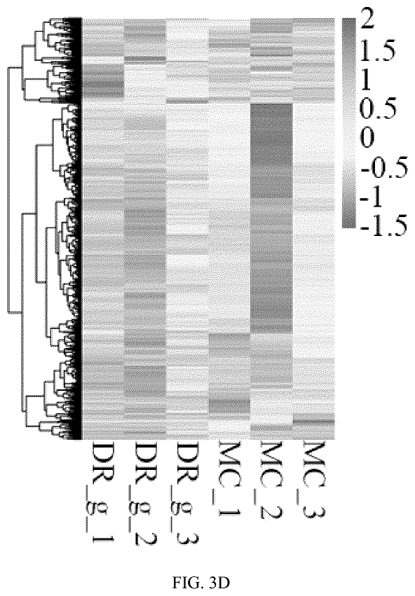

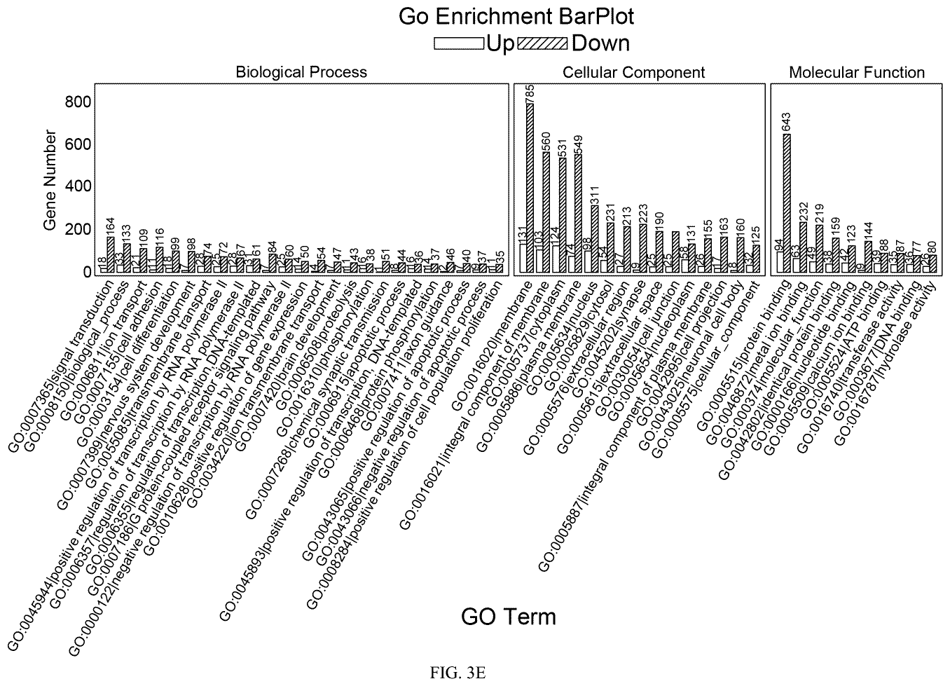

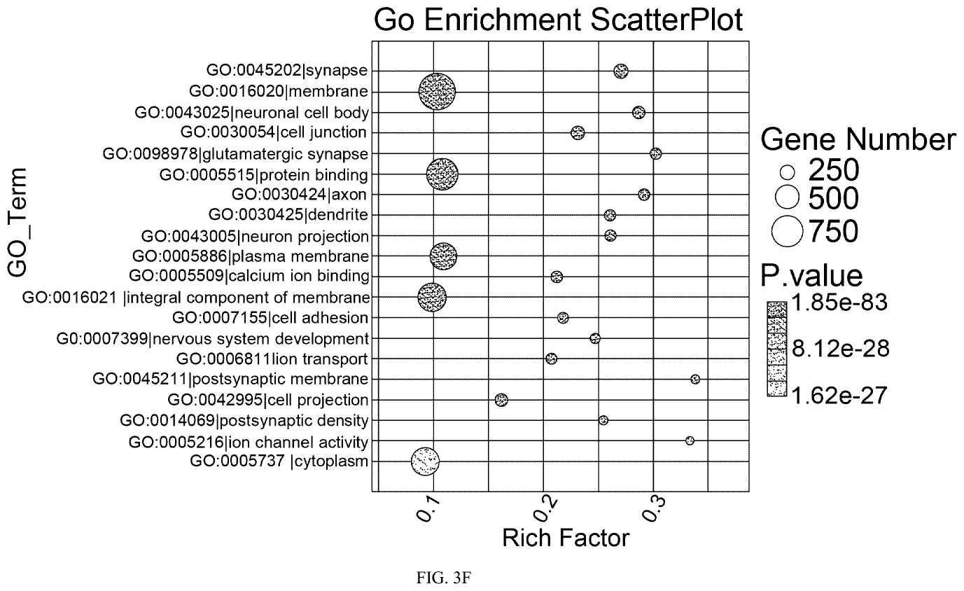

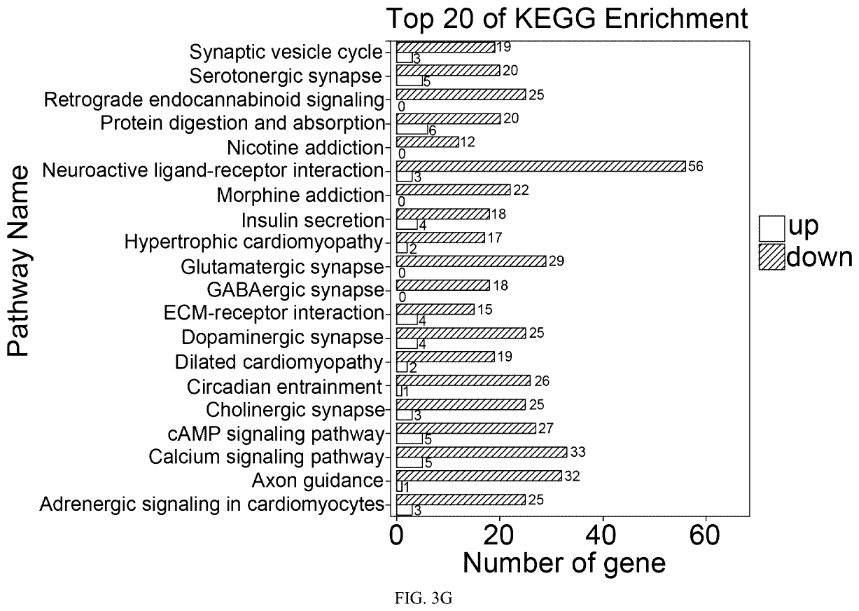

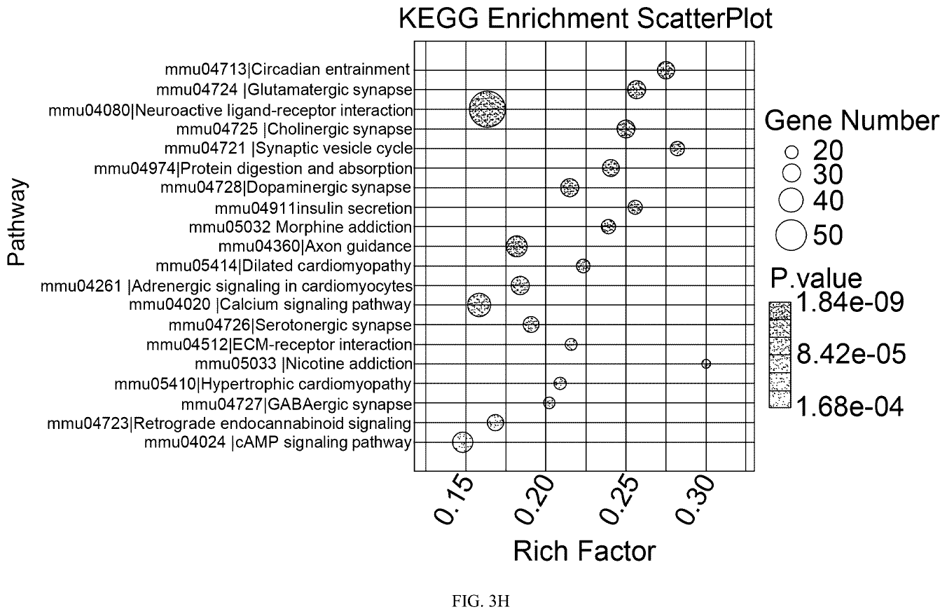

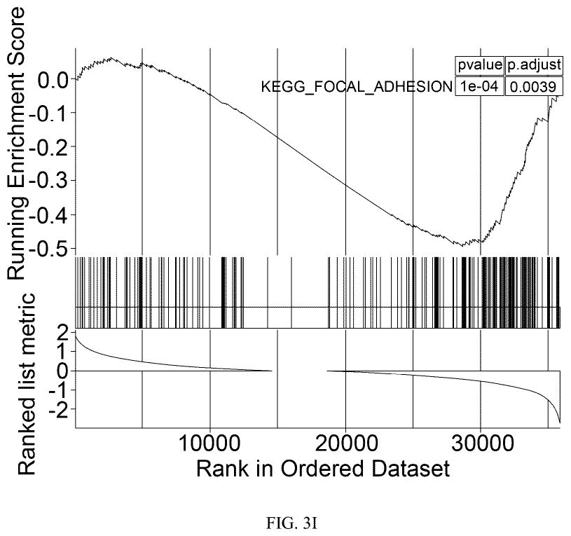

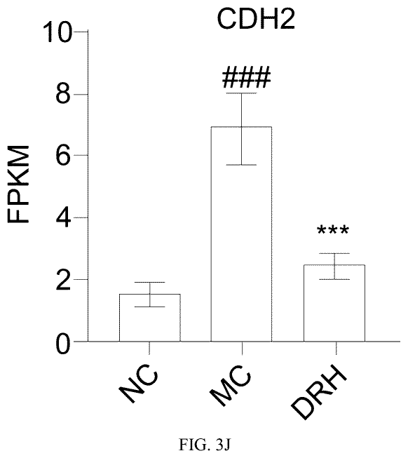

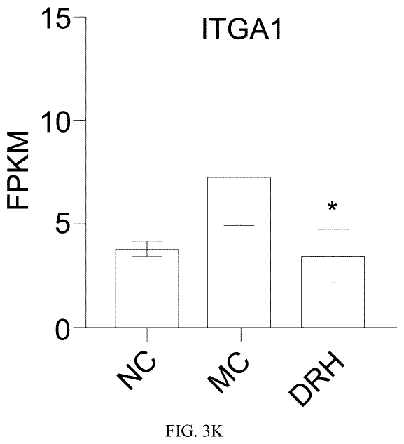

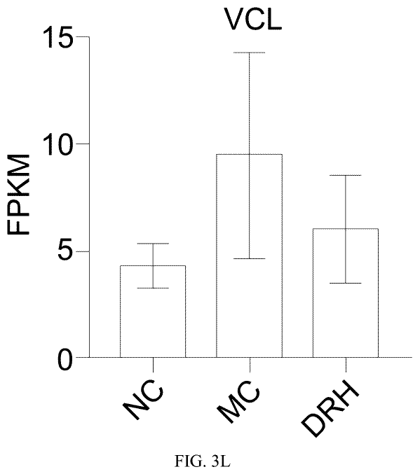

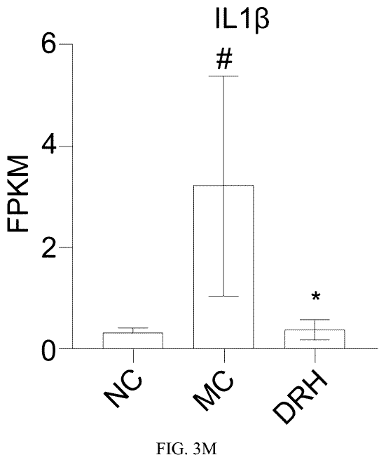

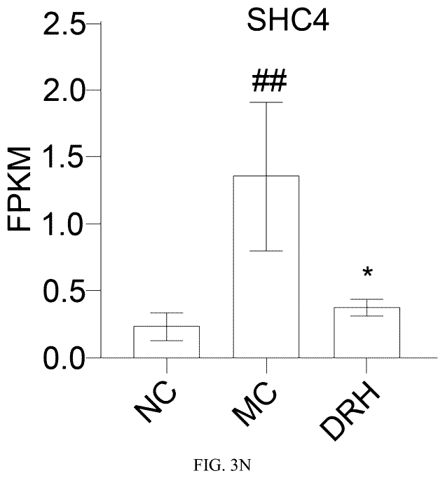

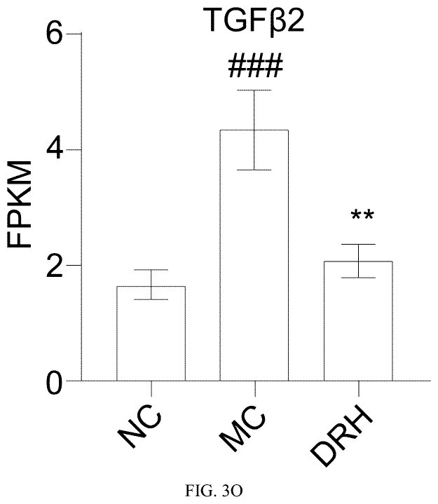

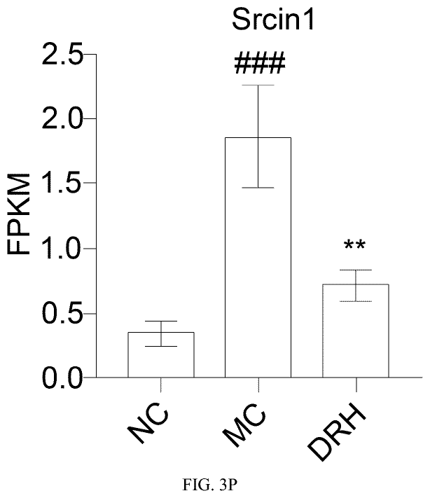

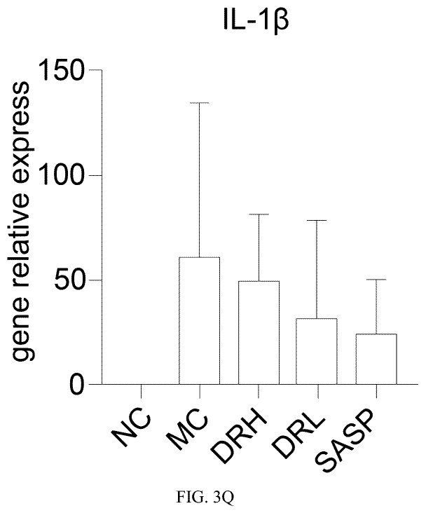

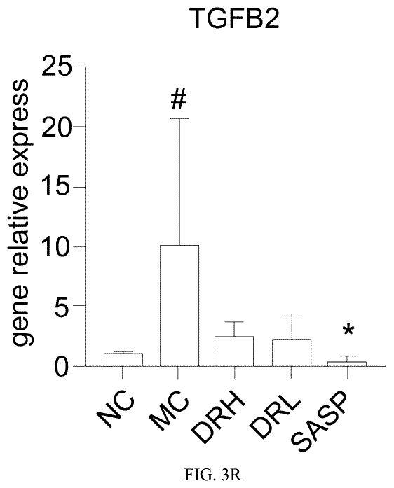

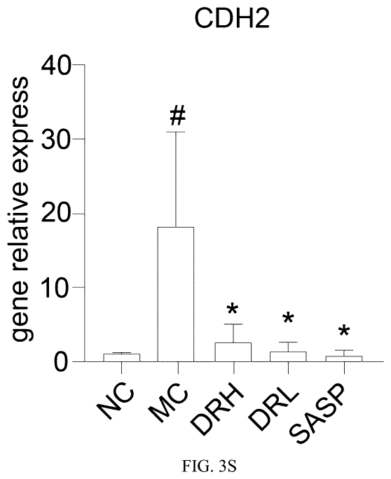

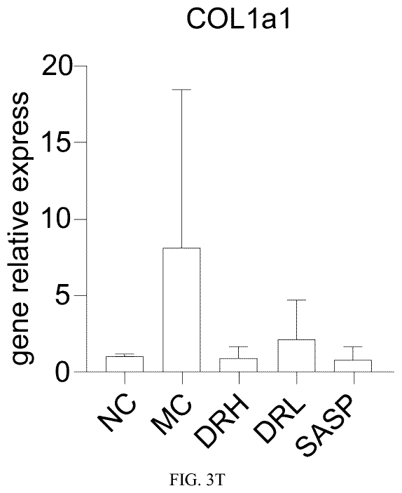

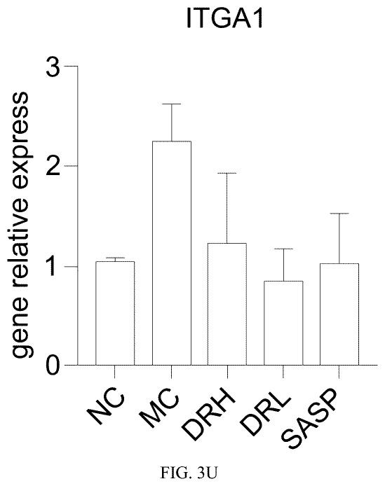

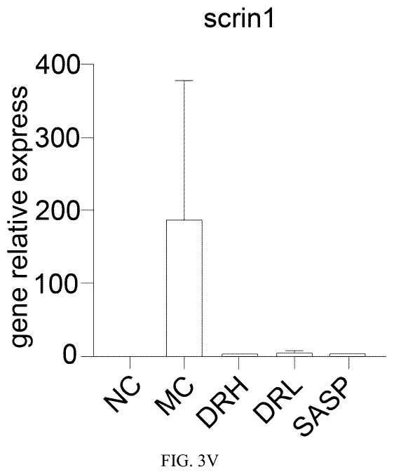

The colon was subjected to RNA-seq, i.e., reference-based transcriptome sequencing, and quantitative analysis of the target genes was performed using reverse transcription quantitative polymerase chain reaction (RT-qPCR). The specific results are shown in FIG. 3.

RNA-seq using reference-based transcriptome sequencing: TRIzol reagent (thermofisher, 15596018) was used to isolate and purify total RNA from the sample; the quantity and purity of RNA were measured using NanoDrop ND-1000 (NanoDrop®, Wilmington, DE, USA), and the integrity of RNA was evaluated using Bioanalyzer 2100 (Agilent®, CA, USA), with concentration >50 ng/μL, RIN>7.0, total RNA>1 μg; the RNA library was prepared and sequenced using the Illumina Novaseq™ 6000 platform from LC-Bio Technologies (Hangzhou) Co., Ltd. (Hangzhou, China); the data were then analyzed and visualized using the R language (www.r-project.org/) and the OmicStudio platform from LC-Bio Technologies (Hangzhou) Co., Ltd. (www.omicstudio.cn/tool); subsequently, the high quality sequencing data were aligned with the reference genome of the project species, and related projects such as gene expression quantification, gene set enrichment analysis (GSEA), gene difference analysis, and enrichment analysis were performed.

Real-time fluorescence quantitative PCR (RT-qPCR): the total RNA was extracted from the colon by using SteadyPure Quick RNA ExtractionKit (AG21023, Accurate Biotechnology, Hunan, China), and stored at −80° C. prior to reverse transcription; reverse transcription was performed using MonScript™ R TIII super Mix with dsDNase reverse transcription kit (MR05201M, Mon ad, Suzhou, China) to synthesize cDNA; RT-qPCR was then performed in a StepOne™ RealTime PCR instrument by using MonAmp™ SYBR Green qPCR Mix (High ROX) (MQ10301S, Monad, Suzhou, China), with target gene primer sequences shown in Table 1; and the procedure included pre-denaturation: 95° C. 3 min; denaturation: 95° C. 10 s; annealing extension: 60° C. 30 s, 40 cycles. The results were calculated using 2^{-ΔΔCT} method to obtain the mRNA relative expression.

TABLE 1
Target gene primer sequences

| Gene | Sequence | Sequence |
|---|---|---|
| TGF- | FORWARD: | SEQ ID |
| beta 2 | TCGACATGGATCAGTTTATGCG | NO. 1 |
| | REVERSE: | SEQ ID |
| | CCCTGGTACTGTTGTAGATGGA | NO. 2 |
| CDH2 | FORWARD: | SEQ ID |
| | AGCGCAGTCTTACCGAAGG | NO. 3 |
| | REVERSE: | SEQ ID |
| | TCGCTGCTTTCATACTGAACTTT | NO. 4 |
| Itgal | FORWARD: | SEQ ID |
| | CCTTCCCTCGGATGTGAGTCA | NO. 5 |
| | REVERSE: | SEQ ID |
| | AAGTTCTCCCCGTATGGTAAGA | NO. 6 |
| ILl-beta | FORWARD: | SEQ ID |
| | GCAACTGTTCCTGAACTCAACT | NO. 7 |
| | REVERSE: | SEQ ID |
| | ATCTTTTGGGGTCCGTCAACT | NO. 8 |
| Srcinl | FORWARD: | SEQ ID |
| | AGCGAGATGCGTTCATGGAC | NO. 9 |
| | REVERSE: | SEQ ID |
| | AACTCCAGTAGTTTGGTTGCTG | NO. 10 |
| collal | FORWARD: | SEQ ID |
| | GCTCCTCTTAGGGGCCACT | NO. 11 |
| | REVERSE: | SEQ ID |
| | CCACGTCTCACCATTGGGG | NO. 12 |

As shown in FIG. 3A, the Pearson correlation coefficient plot demonstrated a good correlation between the samples in the Melastoma dodecandrum Lour. treatment group and the samples in the model group administered with DSS. The PCA (principal component analysis) results showed a significant difference in sample distribution between the Melastoma dodecandrum Lour. group and the model group (FIG. 3B). FIGS. 3C and 3D showed the overall distribution and clustering of differentially expressed genes. Most of the genes in the Melastoma dodecandrum Lour. group were down-regulated compared with those in the model group. Gene ontology (GO) enrichment and kyoto encyclopedia of genes and genomes (KEGG) enrichment (FIGS. 3E-3H) showed that DEGs in the DIREN group versus model group were mainly enriched in “synapse”, “cell junction”, “neuroactive ligand-receptor interaction” and “circadian rhythm entrainment”. Based on the GSEA (gene set enrichment analysis) from KEGG database, it was shown that the genes in the Melastoma dodecandrum Lour. group were enriched in the focal adhesion pathway, and that the gene expression level in the Melastoma dodecandrum Lour. group significantly reduced compared with the model group (FIG. 3I, p.adjust=0.0039). The gene expressions of CDH2, ITGA1 and TGF-beta 2 showed significant inter-group differences (FIGS. 3J-3P). The RT-qPCR analysis of the target genes also confirmed the above results (FIGS. 3Q-3V). Based on the above results, it can be concluded that the extract of Melastoma dodecandrum Lour. of the present disclosure may regulate cell migration, improve intestinal tract barrier, and reduce fibrosis and UC damage through the N-cadherin (encoded by the CDH2 gene) mediated focal adhesion pathway.

### 2.6 Immunohistochemical Staining and Fluorescence Staining

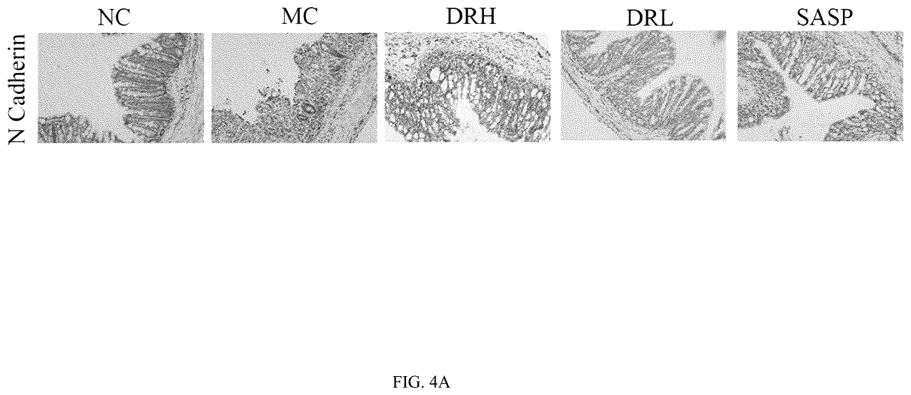

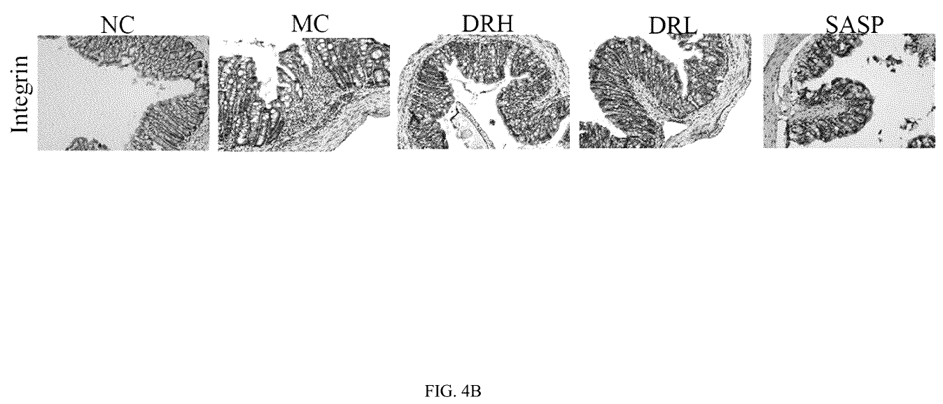

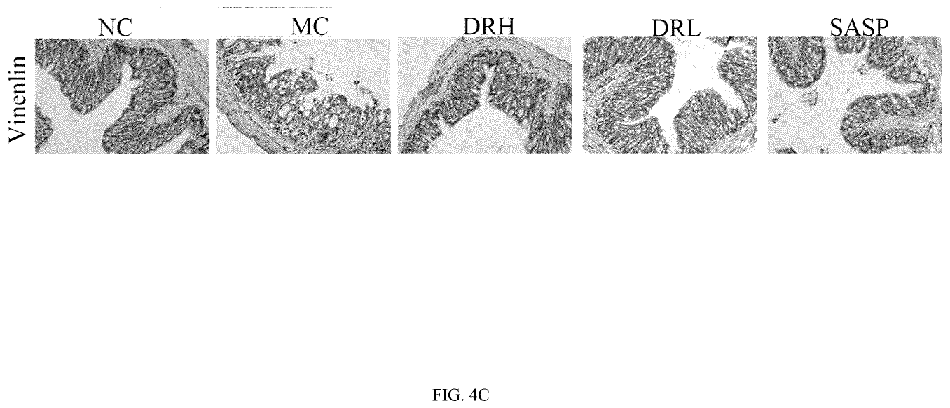

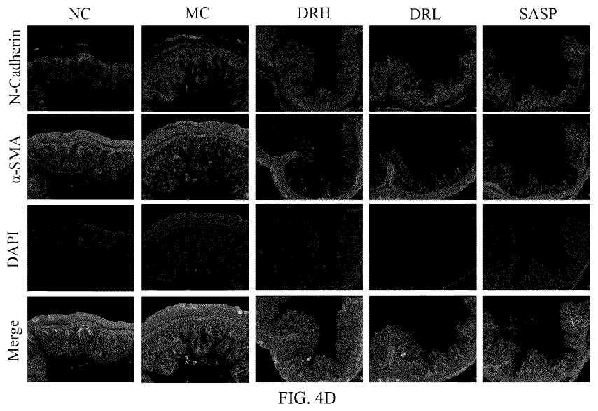

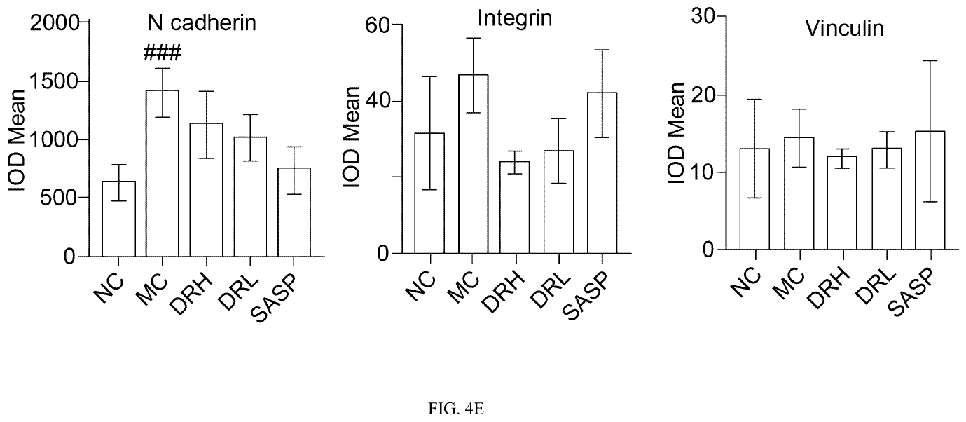

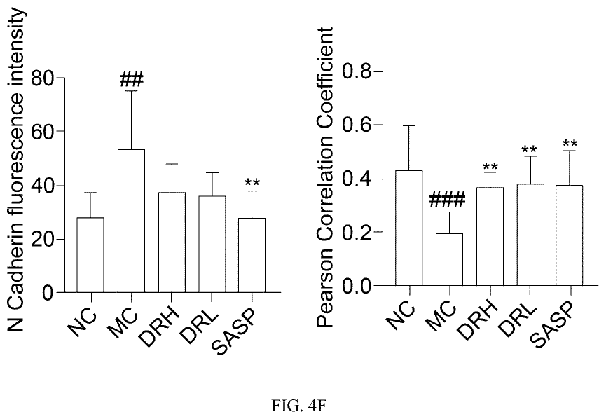

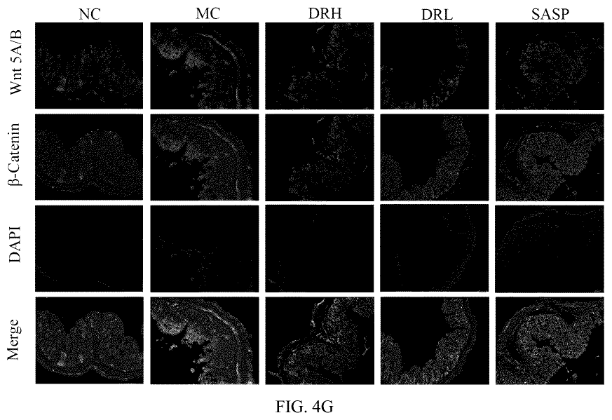

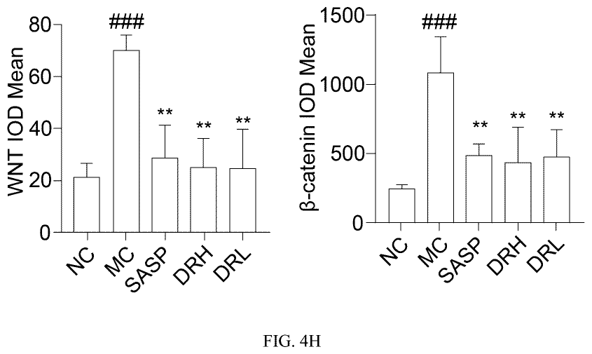

N-cadherin is an adhesive junction that is important for cell attachment, migration, and formation of fibrous connective tissue. To figure out whether there is an association between N-cadherin and fibroblast migration in the colon, the sections of intestinal tissues were subjected to immunohistochemical staining and fluorescence staining. Fibroblasts were labeled with alpha-SMA, a classical marker. Fluorescence co-localization of N-cadherin and intestinal fibroblasts in the intestinal mucosa was analyzed using Pearson Correlation Coefficient (PCC) to explain the correlation of the co-expression of N-cadherin and alpha-SMA. The specific results are shown in FIGS. 4A-4H.

Immunohistochemical staining: the paraffin sections of colon were deparaffinized and rehydrated, and then washed with PBS 3 times; then, microwave retrieval was performed using a sodium citrate solution (pH 6.0, P0081, Beyotime, China); an endogenous peroxide was blocked with 3% hydrogen peroxide; blocking was then performed with an immunostaining blocking solution (P0260, Beyotime®, China) for 15 min at room temperature; the sections were incubated with antibodies N-cadherin (ET1701-39, Huabio, China; dilution 1:200), Integrin A1 (22146-1-AP, Proteintech, China; dilution 1:200), and Vinculin (ET1705-94, Huabio, China; dilution 1:200), respectively, overnight at 4° C.; the corresponding HRP secondary antibody (PV-6001, ZSbio, China) was incubated with the sections for 30 min at 37° C., and then washed with PBS; color development was performed with DAB (ZLI-9019, ZSbio, China), and the sections were subjected to hematoxylin staining, and then rapidly dehydrated in absolute ethanol; and finally, the sections were mounted with neutral resin. Images were taken by an optical microscope, and analyzed using Image-ProPlus 6.0 software.

Immunofluorescence staining: the paraffin sections of colon were deparaffinized and rehydrated, and retrieved with 200 ml of a sodium citrate retrieval solution in a microwave oven, then an endogenous peroxide was blocked with 3% hydrogen peroxide, and the sections were than washed with PBS for 5 min 3 times; the sections were then incubated with N-Cadherin recombinant rabbit monoclonal antibody (SY02-46, Huabio, China) (1:200), alpha-SMA recombinant rabbit monoclonal antibody (ET1607-53, Huabio, China) (1:200), WNT5A/B polyclonal antibody (55184-1-AP, Proteintech, China) (1:200) and beta-catenin (M24002, Abmart, China) (1:200), respectively, overnight at 4° C.; after each incubation, staining was performed according to the instructions of tyramide signal amplification (TSA) double fluorescence staining kit (RK05902, ABclonal, China); and finally, mounting was performed with an antifade mounting medium (with DAPI). The use of TSA fluorescent dyes and all subsequent procedures were performed in the dark. Images were taken using a fluorescence microscope equipped with Zen software (AXIO SCOPE.A1, Carl Zeiss, Germany). Co-localization analysis of fluorescence images was performed using imagepro plus 6.0, with results expressed as Pearson correlation coefficient (PCC).

The immunohistochemical results showed a significant increase in N-cadherin expression in the MC group (FIGS. 4A and 4E, P<0.001). The expressions of IntegrinA1 and Vinculin in the MC group increased, and decreased after the treatment with Melastoma dodecandrum Lour. (FIGS. 4B and 4C). The above RT-PCR results also demonstrated that the gene expressions of TGF-beta 2, integrin, CDH2 (N-cadherin) and Srcin1 in the model group increased, while the expressions decreased after the treatment with Melastoma dodecandrum Lour. (FIG. 3K).

The immunofluorescence results showed that the co-localization in the MC group was significantly lower than that in the NC group or other groups, and that the fluorescence expression of N-cadherin in the MC group also increased (FIGS. 4D, 4F), indicating that N-cadherin leads to fibroblast migration in the MC group. In contrast, Melastoma dodecandrum Lour. improves the co-expression of both and reduces the expression of N-cadherin. These results showed that Melastoma dodecandrum Lour. reduces fibroblast migration by inhibiting DSS-induced overexpression of N-cadherin and focal adhesion pathway proteins (FIGS. 4G and 4H). Activation of the Wnt/beta-Catenin pathway is associated with fibrosis in multiple organs in pathological states. Based on the protein expressions of Wnt 5a/b and beta-Catenin in the colon, it is found that Melastoma dodecandrum Lour. can inhibit DSS-induced protein overexpressions of Wnt 5a/b and beta-Catenin, thereby alleviating intestinal fibrosis. Therefore, the extract of Melastoma dodecandrum Lour. of the present disclosure treats intestinal fibrosis caused by UC through inhibiting the activation of the Wnt/beta-Catenin pathway.

The descriptions above are merely the preferred embodiments of the present disclosure. It should be noted that several improvements and modifications may also be made by those of ordinary skill in the art without departing from the principle of the present disclosure, and these improvements and modifications should also be considered within the protection scope of the present disclosure.

What is claimed is:

1. A method for preparing an extract of Melastoma dodecandrum Lour., comprising: soaking Melastoma dodecandrum Lour. in water, followed by heating to boiling, performing a reflux condensation and filtration, and collecting a filtrate, which constitutes the extract of Melastoma dodecandrum Lour.; wherein the reflux condensation is performed for 0.5 to 1.5 h.

2. The method of claim 1, wherein a mass ratio of the Melastoma dodecandrum Lour. to the water is 1:9 to 11, and the soaking is performed for 25 to 35 min.

3. The method of claim 1, further comprising mixing a medicine residue resulting from the filtration with water after the filtration, performing extraction for 40 to 50 min at 100° C., performing another filtration, and then combining filtrates; a mass ratio of the medicine reside to the water is 1:5 to 9.

4. An extract of Melastoma dodecandrum Lour. obtained by the method of claim 1.

5. The extract of Melastoma dodecandrum Lour. of claim 4, wherein a mass ratio of the Melastoma dodecandrum Lour. to the water is 1:9 to 11, and the soaking is performed for 25 to 35 min.

6. The extract of Melastoma dodecandrum Lour. of claim 4, wherein the method further comprises mixing a medicine residue resulting from the filtration with water after the filtration, performing extraction for 40 to 50 min at 100° C., performing another filtration, and then combining filtrates; a mass ratio of the medicine reside to the water is 1:5 to 9.

7. A method for treating ulcerative colitis (UC), comprising administering to a subject in need thereof a therapeutically effective amount of the extract of Melastoma dodecandrum Lour. of claim 4.

8. A method for treating intestinal fibrosis, comprising administering to a subject in need thereof a therapeutically effective amount of the extract of Melastoma dodecandrum Lour. of claim 4.

9. The method of claim 7, wherein the extract of Melastoma dodecandrum Lour. reduces colonic epithelial collagen deposition and intestinal epithelial cell apoptosis.

10. The method of claim 8, wherein the extract of Melastoma dodecandrum Lour. reduces colonic epithelial collagen deposition and intestinal epithelial cell apoptosis.

11. The method of claim 7, wherein the extract of Melastoma dodecandrum Lour. has an effect of down-regulating the abnormally high expression of genes and proteins such as N-cadherin, IntegrinA1, Vinculin, TGF-β2 and Srcin in colonic tissue.

12. The method of claim 8, wherein the extract of Melastoma dodecandrum Lour. has an effect of down-regulating the abnormally high expression of genes and proteins such as N-cadherin, IntegrinA1, Vinculin, TGF-β2 and Srcin in colonic tissue.

13. The method of claim 7, wherein the extract of Melastoma dodecandrum Lour. has effects of regulating fibroblast migration, reducing colonic fibrosis and inflammatory damage, and protecting goblet cells and colonic epithelial barrier function.

14. The method of claim 8, wherein the extract of Melastoma dodecandrum Lour. has effects of regulating fibroblast migration, reducing colonic fibrosis and inflammatory damage, and protecting goblet cells and colonic epithelial barrier function.

15. The method of claim 7, wherein the medicament comprises the extract of Melastoma dodecandrum Lour. and a pharmaceutically acceptable excipient.

16. The method of claim 8, wherein the medicament comprises the extract of Melastoma dodecandrum Lour. and a pharmaceutically acceptable excipient.


SEQUENCE LISTING (12 sequences)

SEQ ID NO: 1 - DNA, 22 bp, synthetic construct
  source 1..22: mol_type = other DNA; note = Forward primer for TGF-beta 2
tcgacatggatcagtttatgcg

SEQ ID NO: 2 - DNA, 22 bp, synthetic construct
  source 1..22: mol_type = other DNA; note = Reverse primer for TGF-beta 2
ccctggtactgttgtagatgga

SEQ ID NO: 3 - DNA, 19 bp, synthetic construct
  source 1..19: mol_type = other DNA; note = Forward primer for CDH2
agcgcagtcttaccgaagg

SEQ ID NO: 4 - DNA, 23 bp, synthetic construct
  source 1..23: mol_type = other DNA; note = Reverse primer for CDH2
tcgctgctttcatactgaacttt

SEQ ID NO: 5 - DNA, 21 bp, synthetic construct
  source 1..21: mol_type = other DNA; note = Forward primer for Itgal
ccttccctcggatgtgagtca

SEQ ID NO: 6 - DNA, 22 bp, synthetic construct
  source 1..22: mol_type = other DNA; note = Reverse primer for Itgal
aagttctccccgtatggtaaga

SEQ ID NO: 7 - DNA, 22 bp, synthetic construct
  source 1..22: mol_type = other DNA; note = Forward primer for ILl-beta
gcaactgttcctgaactcaact

SEQ ID NO: 8 - DNA, 21 bp, synthetic construct
  source 1..21: mol_type = other DNA; note = Reverse primer for ILl-beta
atcttttggggtccgtcaact

SEQ ID NO: 9 - DNA, 20 bp, synthetic construct
  source 1..20: mol_type = other DNA; note = Forward primer for Srcinl
agcgagatgcgttcatggac

SEQ ID NO: 10 - DNA, 22 bp, synthetic construct
  source 1..22: mol_type = other DNA; note = Reverse primer for Srcinl
aactccagtagtttggttgctg

SEQ ID NO: 11 - DNA, 19 bp, synthetic construct
  source 1..19: mol_type = other DNA; note = Forward primer for collal
gctcctcttaggggccact

SEQ ID NO: 12 - DNA, 19 bp, synthetic construct
  source 1..19: mol_type = other DNA; note = Reverse primer for collal
ccacgtctcaccattgggg